(19) United States
(12) Patent Application Publication
(10) Pub. No.: US 2002/0115592 A1
(43) Pub. Date: Aug. 22, 2002

(54) Pharmaceutical compositions containing insulin

(75) Inventors: Roger C. New, London (GB); John R. Langridge, Dembigshire (GB); Christopher J. Smith, Dembighshire (GB)

Correspondence Address: STERNE, KESSLER, GOLDSTEIN & FOX PLLC, 1100 NEW YORK AVENUE, N.W., SUITE 600, WASHINGTON, DC, 20005-3934, US

(73) Assignee: Provalis UK Limited

(21) Appl. No.: 09/871,906

(22) Filed: Jun. 4, 2001

Related U.S. Application Data
(63) Continuation of application No. PCT/GB99/04067, filed on Dec. 3, 1999.

(30) Foreign Application Priority Data
Dec. 4, 1998 (GB) GB 9826821.2
Dec. 4, 1998 (GB) GB 9826822.0

(51) Int. Cl. A61K 38/28; A61K 31/17

(52) U.S. Cl. 514/3; 514/12

(57) ABSTRACT

A pharmaceutical composition comprising insulin and optionally aprotinin, in a substantially non-aqueous hydrophilic medium comprising an alcohol and a cosolvent, in association with a medium chain partial glyceride, optionally in admixture a long-chain PEG species.

### CROSS-REFERENCE TO RELATED APPLICATIONS

The present application is a continuation of the International Application, PCT/GB 99/04067, filed Dec. 3, 1999, published in the English language, which claims priority benefit to Great Britain applications GB 9826821.2, filed Dec. 4, 1998 and GB 9826822.0, filed Dec. 4, 1998. The full disclosures of each of these applications are herein incorporated by reference.

### FIELD OF THE INVENTION

The present invention relates to novel oil-based pharmaceutical compositions containing insulin. The invention also relates to methods of preparing such compositions.

In order for therapeutic agents to display maximum efficacy, they often need to be presented to the body in a finely dispersed form, or in a form which will result in rapid dispersion in the body. The simplest way to achieve this is by administering such agents in liquid form as solutions. While solutions in aqueous media are the most readily formed, and convenient to manufacture, formulations which contain significant quantities of free water may suffer from the disadvantage of reduced shelf-life and limited long-term storage stability. This may particularly be the case with proteins, where the small water molecules can penetrate into the interior of proteins and induce conformational changes which, in conjunction with formation and reshuffling of hydrogen bonds, can result in denaturation of the protein, and loss of activity. A further disadvantage is that solutions comprising an aqueous component may often be incompatible with other potential excipients of a pharmaceutical formulation (e.g. oils, or materials whose action is manifested in the dry form).

Consequently, the availability of solutions of hydrophilic therapeutic agents in non-aqueous solvents would be highly desirable. An example of such a medium would be a polyethylene glycol e.g. PEG 200. Other examples are glycerol, propanediol (also named propylene glycol), tetraglycol and transcutol. Unfortunately, the ability of insulin to dissolve in these media, at high concentration, without the intermediary of an aqueous phase, is surprisingly limited.

Furthermore, insulin is difficult to dissolve in hydrophilic solvents, even when water is present. In general only low concentrations of insulin are achieved and the dissolution time may be too long to be of use in a viable manufacturing process.

The difficulties of formulating insulin are well known in the art and numerous attempts have been made to overcome them.

Thus, for example, WO 95/13795 describes single phase preparations of hydrophilic species such as insulin in a hydrophobic phase, which preparations are obtained by associating the hydrophilic species with an amphiphile in a liquid medium, removing the liquid medium to leave an array of amphiphile and hydrophilic molecules and then providing a hydrophobic solvent around the array. The liquid medium used may be a polar organic solvent such as dimethylformamide, dimethylsulphoxide or glacial acetic acid, or it may be water. In the latter case the water may be removed by freeze-drying.

WO 96/17593 describes similar compositions which are prepared using a solubilisation aid selected from (a) a low molecular weight compound having at least some degree of polarity, (b) a lipid-soluble organic acid (c) an amphiphile and (d) glycerol or other polyhydric alcohol. Both (a) and (b) may be inter alia a carboxylic acid. However, as in WO 95/13795, a key step is the removal of the liquid medium.

The need to remove the liquid medium requires a separate step and introduces additional complexity in a manufacturing process. It would therefore be desirable to prepare formulations which did not require such a step.

U.S. Pat. No. 5,284,657 (Abbot Laboratories) describes pharmaceutical compositions for the sublingual or buccal administration of therapeutic agents which are normally degraded upon oral administration, especially polypeptides (inter alia insulin), comprising in addition to the therapeutic agent a solvent system comprising a non-toxic alcohol (inter alia propylene glycol or polyethylene glycol) and an oral mucosal membrane transport enhancing agent which may be an acid (inter alia lactic acid) or an essential or volatile oil. The preferred polypeptide is leuprolide, the preferred alcohol is ethanol and the preferred acid is benzoic acid. There are no specific examples of formulations containing insulin. The formulations may optionally contain a co-solvent selected from water or a pharmaceutically acceptable oil. The non-toxic alcohol comprises about 50-95% w/v of the total volume of the carrier, the transport enhancing agent about 0.5-50% w/v and the co-solvent, when present, about 5 to 50% w/v. There is no suggestion in this patent that any of the formulations could be used for oral administration, i.e. for absorption from the digestive tract.

U.S. Pat. No. 5,206,219 (Applied Analytical Industries Inc.) describes enteric coated pharmaceutical compositions adapted for oral administration which comprise a proteinaceous medicament, which may be inter alia insulin, formulated in a medium comprising a pharmaceutical solvent such as polyethylene glycol or propylene glycol and a lipid pharmaceutical solvent such as oleic acid. Typically, the amounts of these co-solvents are about 15-35% of polyol to 30-60% of lipid. The polyol solvent phase may also contain inter alia an organic acid, e.g. citric acid, as a stabiliser as well as a high HLB surfactant. The lipid phase may also contain additional ingredients, such as cholesterol, a phospholipid and a lipophilic surfactant. The compositions are said to be in the form of a clear liquid which may range from free-flowing to slightly viscous in nature.

UK patent application GB2142238 (Nitto Electric Industrial Co.) describes pharmaceutical competitions for percutaneous administration which comprise a pharmaceutical agent such as a benzodiazepine in a carrier comprising three components, the first of which (A) may be selected from an optionally halo-substituted aliphatic hydrocarbon; an aliphatic carboxylic acid ester; an ether; a ketone; or an aliphatic mono-alcohol, all of which are hydrophobic in nature. The second component (B) may be inter alia lactic acid and the third component (C) is a diol. The weight of (A) is 0.1-80% of (A) +(B) and the weight of (C) is preferably 10-100 wt % of (B).

WO 98/00155 (University of Utah Research Foundation) describes both aqueous liquid compositions of calcitonin comprising an aqueous mixture of SDS and an organic acid and non-aqueous liquid compositions of calcitonin comprising about 90-100% by volume of a mixture of C₈/C₁₀ mono-and di-glycerides and about 0-10% by volume of a polar, non-aqueous solvent.

However, there remains a need for improved formulations of insulin.

We have now surprisingly found that it is possible to dissolve insulin and optionally aprotinin in a non-aqueous hydrophilic solvent in unusually high concentrations and in a relatively short period of time. We have further found that the resulting solutions may be admixed with a hydrophobic solvent such as a medium chain partial glyceride to provide a composition which is particularly advantageous for pharmaceutical administration, especially by the oral route. The compositions according to the invention are preferably in the form of a fine suspension.

Compositions according to the present invention (as defined hereinafter) have been found to have a number of advantages. Thus, they may be manufactured in a small number of relatively straightforward process steps, avoiding the need for freeze-drying or other techniques for removal of water. They generally contain fewer ingredients and are thus less complex to prepare than many of the aforementioned formulations. When the compositions according to the invention are obtained as suspensions, these are stable suspensions having a consistent and reproducible particle size. The provision of insulin in the form of a fine suspension differs from formulations known in the art, such as micro-emulsions and has advantages over such formulations e.g. in terms of stability. Furthermore, the compositions provide a high concentration of insulin and demonstrate good levels of absorption of insulin following oral administration. This is a particularly surprising finding in view of the fact that compositions of insulin simply suspended in an oil phase, without a non-aqueous hydrophilic medium do not have the requisite activity for pharmaceutical use.

In a first aspect therefore, the present invention provides a pharmaceutical composition comprising a solution of insulin optionally in admixture with aprotinin, in a substantially non-aqueous hydrophilic medium comprising an alcohol and a cosolvent, in association with a medium chain partial glyceride, optionally in admixture with a long-chain PEG species.

In a second aspect the present invention provides a method for preparing a pharmaceutical formulation comprising insulin optionally in admixture with aprotinin, in accordance with the first aspect which method comprises the steps of:

(a) dissolving said insulin and optionally aprotinin in a substantially non-aqueous hydrophilic medium comprising an alcohol, and a co-solvent and

(b) bringing the resulting solution into association with a medium chain partial glyceride, if necessary or desired, in admixture with a long-chain PEG species.

An alcohol for use in the present invention may include for example a C₂-C₈ monohydric alcohol such as ethanol, n-propanol, isopropanol or t-butanol; a C₂-C₈ polyhydric alcohol, eg a glycol such as ethylene glycol (1,2-ethanediol), propylene glycol (1,2-propanediol), trimethylene glycol (1,3-propanediol) or glycerol (1,2,3-propanetriol) or an ether or polyether terminating in one or two hydroxyl groups such as polyethylene glycol, tetraglycol or transcutol. It will be appreciated that when a polyethylene glycol is used as solvent it should be a liquid. In general, polyethylene glycols with a molecular weight of less than 600 Daltons will be suitable. Thus, preferred polyethylene glycols include PEG 200, PEG 300 and PEG 400. The alcohol is preferably a liquid polyethylene glycol, eg PEG 300, or a polyhydric alcohol, such as glycerol or propylene glycol or a mixture thereof. Polyethylene glycols are available under a variety of trade names, for example, PEG 300 is available under the name Macrogol 300™.

The cosolvent may be an acid such as a carboxylic acid or a sulphonic acid; a salt of a weak acid such as sodium acetate or sodium ursodeoxycholate; a weak base such as triethylamine; or a zwitterionic compound such as carnitine.

A carboxylic acid for use in the present invention may be for example a C₂-C₈ alkylcarboxylic acid, optionally substituted by OH and carrying 1, 2, or 3 carboxyl groups. Examples of such carboxylic acids include acetic acid, lactic acid, citric acid, caproic acid or malic acid. The acid is preferably lactic acid or acetic acid. A particularly preferred acid is DL-lactic acid which is a liquid at room temperature.

A sulphonic acid for use in the present invention may be for example benzene sulphonic acid, toluene-sulphonic acid or methane-sulphonic acid.

In a preferred embodiment the hydrophilic medium contains both acidic and hydroxyl functions. These may be provided by admixture of both a carboxylic acid and an alcohol as defined above. Alternatively the acid and hydroxyl functions may both be provided by the same compound, e.g. lactic acid. DL-lactic acid is a particularly preferred cosolvent for use according to the present invention.

The components of the hydrophilic medium should be selected so as to dissolve the insulin (and aprotinin, if present). Preferably, complete dissolution of the said macromolecule(s) is achieved. The hydrophilic medium may contain more than one alcohol and/or more than one cosolvent. It will be appreciated that the solvent(s) and cosolvent(s) comprising the non-aqueous hydrophilic dissolution medium should be miscible.

It has also been found that DL-lactic acid alone (i.e. without admixture with an alcohol) can provide a suitable non-aqueous hydrophilic dissolution medium for insulin and optionally aprotinin. In a further aspect therefore, the present invention provides a pharmaceutical composition comprising a solution of insulin, optionally in admixture with aprotinin, in DL-lactic acid, in association with a medium chain partial glyceride, optionally in admixture with a long-chain PEG species.

The present invention also provides a method for preparing a pharmaceutical formulation comprising insulin optionally in admixture with aprotinin, in accordance with the first aspect which method comprises the steps of:

(a) dissolving said insulin and optionally aprotinin in DL-lactic acid and

(b) bringing the resulting solution into association with a medium chain partial glyceride, if necessary or desired, in admixture with a long-chain PEG species.

Thus, particular non-aqueous hydrophilic dissolution media according to the present invention include:

an alcohol such as a polyethylene glycol, tetraglycol or transcutol admixed with a C₂-C₈ monohydric alcohol or a C₂-C₈ polyhydric alcohol, and a carboxylic acid or sulphonic acid or a salt such as sodium ursodeoxycholate or sodium acetate;

an alcohol such as a polyethylene glycol, tetraglycol or transcutol admixed with a C₂-C₈ monohydric alcohol or a C₂-C₈ polyhydric alcohol and a weak base such as triethylamine or a zwitterionic compound such as carnitine;

an alcohol such as a polyethylene glycol, tetraglycol or transcutol, admixed with a carboxylic acid or sulphonic acid or with a salt such as sodium ursodeoxycholate, or sodium acetate;

an alcohol such as a polyethylene glycol, tetraglycol or transcutol, admixed with a zwitterionic compound such as carnitine;

an C₂-C₈ polyhydric alcohol such as glycerol or propanediol admixed with a carboxylic acid or sulphonic acid or with a salt such as sodium ursodeoxycholate, sodium acetate or sodium L-lactate;

DL-lactic acid.

Suitable dissolution media therefore include:

a polyethylene glycol+propanediol or glycerol+acetic acid, citric acid, lactic acid or caproic acid;

a polyethylene glycol+1,2-propanediol, 1,3-propanediol or glycerol+DL-lactic acid;

a polyethylene glycol+1,2-propanediol, 1,3-propanediol or glycerol+triethylamine or carnitine;

a polyethylene glycol+citric acid, lactic acid or caproic acid;

a polyethylene glycol+DL-lactic acid;

a polyethylene glycol+carnitine;

a polyethylene glycol+1,2-propanediol, 1,3-propanediol or glycerol+sodium ursodeoxycholate, or sodium acetate;

a polyethylene glycol+sodium ursodeoxycholate or sodium acetate; 1,2-propanediol, 1,3-propanediol or glycerol+acetic acid, citric acid, lactic acid or caproic acid;

1,2-propanediol, 1,3-propanediol or glycerol+sodium ursodeoxycholate, sodium acetate or sodium L-lactate; and

DL-lactic acid.

A preferred hydrophilic dissolution medium according to the present invention is a mixture of polyethylene glycol and DL-lactic acid.

In the compositions according to the present invention the medium chain glyceride is preferably selected from mono and diglycerides (partial glycerides), and advantageously is a mixture of medium chain mono- and di-glycerides. Suitably, medium chain partial glycerides for use in the present invention have chain lengths of 8 to 10 carbon atoms, for example they can comprise straight chain saturated fatty acids. In particular monoglycerides can make up 40-90% of the total amount of the oil phase, preferably 60-70%. Examples of a suitable mixture of glycerides include some grades of Akoline™ (available from Karlshamns Sweden AB, S/34782 Karlshamn, Sweden) and some grades of Imwitor™ (Condea, Germany). These products contain predominantly mixtures of mono- and di-glycerides of capric (C₁₀) and caprylic (C₈) acids.

The medium chain partial glyceride preferably comprises at least 80% of the composition, preferably 85% and advantageously 90%. The hydrophilic phase preferably comprises no more than 20% of the total composition, preferably no more than 15% and advantageously no more than 10%.

In certain embodiments of the present invention it may be necessary or desirable to add to the medium chain glyceride a long-chain PEG species, such as polyoxyethylene-40-monostearate (POE-40-S) or PEG 3350. This has been found to aid formation of a suspension. It has been found however, that in general insulin will form a suspension without the addition of such material.

The medium chain partial glyceride may if desired contain agents which are known in the pharmaceutical art e.g. to aid dispersion in vivo. Thus for example the glyceride may contain a surfactant, such as polyoxyethylated castor oil derivatives or other POE-containing surfactants.

Compositions according to the present invention can be prepared by dissolving insulin and optionally aprotinin in a substantially non-aqueous hydrophilic medium as defined above, eg a mixture of polyethylene glycol and DL-lactic acid, and mixing the resulting solution with a medium chain partial glyceride as defined above optionally admixed with a long-chain PEG species. On mixing, insulin may precipitate out of the hydrophilic medium to form a fine suspension. The mixing of the components may be carried out by conventional methods. Thus for example the macromolecule solution may be added to the partial glyceride with stirring.

We have found that the insulin so formed in the compositions according to the invention has a smaller particle size than a sample of insulin suspended directly in a medium chain glyceride. Furthermore the compositions are found to have a consistent and relatively narrow particle size range. Thus, 50% by weight of the particles have a diameter (D₅₀) of less than 30 micron preferably less than 5 micron, eg between 0.5 and 5.0 micron. This range has previously been reported to be particularly advantageous for uptake of substances from the gut.

It has further been found that 90% by weight of particles in compositions according to the present invention have a diameter (D₉₀) of less than 50 micron, preferably less than 30 micron, eg from 1 to 30 micron.

A preferred composition according to the present invention comprises a fine suspension of insulin which is formed by bringing into association:

(a) a medium chain partial glyceride and

(b) a solution of insulin in a substantially non-aqueous hydrophilic medium comprising one or more solvents selected from a polyethylene glycol, tetraglycol, transcutol and a polyhydric alcohol and a cosolvent which is a carboxylic acid, preferably such that the medium provides an acidic function and a hydroxyl function;

wherein component (a) comprises at least 90% and component (b) comprises no more than 10% of the total composition.

As indicated above, the compositions according to the present invention allow formulation of insulin without the presence of water, which is highly advantageous. Thus the compositions will in general, preferably be non-aqueous. However, in some instances the hydrophilic phase may, unavoidably or by design include a small amount of water and the present invention does not preclude this. Preferably, the amount of water used is no more than 5% of the total composition.

A further advantage of compositions according to the present invention is that they enable insulin to be dissolved in a non-aqueous hydrophilic medium in relatively high concentrations. Thus, insulin is preferably present in the hydrophilic phase in a concentration of at least 75 mg/ml, most preferably at least 100 mg/ml and in some cases at least 400 mg/ml.

In a further aspect the present invention provides insulin obtainable by the steps of

(a) dissolving insulin in a substantially non-aqueous hydrophilic medium as defined above, eg a mixture of polyethylene glycol and DL-lactic acid;

(b) mixing the resulting solution with a medium chain partial glyceride as defined above;

(c) precipitating a fine suspension of insulin; and

(d) isolating said insulin.

Compositions according to the present invention have been found to give good absorption of insulin following administration to the duodenum of pigs.

The compositions according to the present invention may be utilised in a number of ways. Thus they may be used directly for administration to a human or animal subject, or they may be further adapted for particular means and routes of administration.

Advantageously compositions according to the invention are adapted for administration as liquid formulations. Thus they may be filled into capsules, for example hard or soft gelatin capsules, which may optionally be enterically coated.

The compositions may be formulated to contain and administered to provide levels of insulin which are within the conventional dosage ranges for this compound. It is envisaged that the compositions according to the present invention will be applicable for the formulation of other hydrophilic macromolecules, in particular proteins and polysaccharides. It will therefore be appreciated that the present invention is not limited to compositions of insulin and aprotinin, but extends to compositions of other hydrophilic macromolecules which will form fine suspensions under the conditions described hereinabove.

In some circumstances, the hydrophilic phase described above may itself be used as a pharmaceutical composition, without admixture in a hydrophobic phase. It is believed that the hydrophilic phase described above itself represents a novel pharmaceutical composition.

Thus in a further aspect the present invention provides a pharmaceutical composition comprising a solution of insulin and optionally aprotinin in a substantially non-aqueous hydrophilic medium comprising an alcohol and a cosolvent, as defined above.

Preferred hydrophilic compositions according to the present invention comprise a solution of insulin in the particular and specific hydrophilic media listed above, most preferably a solution of insulin in polyethylene glycol and DL-lactic acid.

The hydrophilic compositions according to the present invention may be used directly for administration to a human or animal subject, or they may be further adapted for particular means and routes of administration.

### Experimental

### Formulation

Compositions according to the invention were prepared as follows:

| | g/batch | mg/capsule |
|---|---|---|
| Insulin (human)* | 7.435 | 4.089 |
| Mono- and di-glycerides | 933.56 | 512.9 |
| D,L-Lactic acid | 30 | 16.50 |
| Macrogol 300 ™ | 30 | 16.50 |

*based on a typical potency of 26.9 iu/mg.

1. The insulin was dissolved in a mixture of the D,L-lactic acid and the Macrogol 300™, and the resulting solution was mixed with the mono- and di-glycerides to form a fine dispersion.

2. The resulting dispersion was filled into white, opaque soft gelatin capsules. The capsules were dried until water content of the shell was less than 14%.

3. The capsules were coated with an enteric coating solution to a target weight of 142 mg per capsule.

### Particle Size

Dispersions were diluted with Akoline™ and the particle size of the suspended insulin was determined by laser light scattering, using a Malvern Mastersizer™.

In typical batches of insulin suspensions prepared according to the above method (to stage 1), the insulin was found to have the particle sizes shown in Table 1, Examples A-H:

TABLE 1
Particle Size data of Insulin Dispersion Formulations

| Formulation | Batch Reference | | | | | | | | |
|---|---|---|---|---|---|---|---|---|---|
| (mg/g) | A | B | C | D | E | F | G | H | I |
| Insulin | 7.435 | 7.435 | 7.435 | 7.435 | 7.435 | 7.435 | 7.435 | 7.435 | QS |
| Macrogol | 40.00 | 30.00 | 40.00 | 20.00 | 30.00 | 40.00 | — | — | — |
| 200 | | | | | | | | | |
| Macrogol | — | — | — | — | — | — | 30.00 | 40.00 | — |
| 300 | | | | | | | | | |
| Lactic Acid | 10.00 | 20.00 | 20.00 | 30.00 | 30.00 | 30.00 | 30.00 | 30.00 | — |
| MDG | 942.6 | 942.6 | 932.6 | 942.6 | 932.6 | 922.6 | 932.6 | 922.6 | QS |
| D₁₀ (micron) | 3.34 | 0.34 | 0.40 | 0.34 | 0.35 | 0.34 | 0.34 | 0.34 | 4.93 |
| D₅₀ (micron) | 21.94 | 3.10 | 3.44 | 2.44 | 1.03 | 0.84 | 0.75 | 0.75 | 35.83 |
| D₉₀ (micron) | 38.24 | 17.07 | 25.61 | 12.74 | 3.33 | 2.73 | 2.71 | 2.74 | 57.12 |

Note: 7.435 mg of insulin of potency 26.9 iu is equivalent to 200 iu.

The following formulations were also prepared:

### Insulin

| Insulin | | Solvent | | Cosolvent | | Addition of |
|---|---|---|---|---|---|---|
| Amount | | (amount) | | (amount) | Soluble? | Akoline |
| 7 | mg | Gly (40 | μl) | Acetic acid | Yes | Opaque |

| | |
|---|---|
| (10 | mg) |

| | | | | | | |
|---|---|---|---|---|---|---|
| 7 | mg | PG (40 | μl) | Citric acid | Yes | Translucent |

| | |
|---|---|
| (10 | mg) |

| | | | | | | |
|---|---|---|---|---|---|---|
| 7 | mg | PG (40 | μl) | Acetic acid | Yes | Translucent |

| | |
|---|---|
| (10 | mg) |

| | | | | | | |
|---|---|---|---|---|---|---|
| 7 | mg | PG (40 | μl) | Caproic acid | Yes | Translucent |

| | |
|---|---|
| (10 | mg) |

| | | | | | | |
|---|---|---|---|---|---|---|
| 7 | mg | PEG (50 | μl) | Glycerol/TEA | Yes | Suspension |

| | | |
|---|---|---|
| (50 | μl/10 gl) | formed |

| | | | | | | |
|---|---|---|---|---|---|---|
| 7 | mg | PEG (40 | μl) | camitine | Yes | Clear solution |

| | |
|---|---|
| (10 | mg) |

| | | | | | | |
|---|---|---|---|---|---|---|
| 7 | mg | PEG (25 | μl) | DL-lactic acid | Yes | Cloudy |

| | |
|---|---|
| (25 | μl) |

| | | | | | | |
|---|---|---|---|---|---|---|
| 7 | mg | PEG (25 | μl) | DL-lactic acid | Yes | Suspension |

| | | |
|---|---|---|
| (25 | μl) | (Akoline/POE) |

| | | | | | | |
|---|---|---|---|---|---|---|
| 7 | mg | Gly (40 | μl) | NaAc | Yes | Translucent |

| | |
|---|---|
| (10 | mg) |

| | | | | | | |
|---|---|---|---|---|---|---|
| 7 | mg | Gly (40 | μl) | L-lactic acid | Yes | Turbid |

| | |
|---|---|
| (10 | mg) |

| | | | | | | |
|---|---|---|---|---|---|---|
| 7 | mg | PD (40 | μl) | NaAc | Yes | Clear |

| | |
|---|---|
| (10 | mg) |

| | | | | | | |
|---|---|---|---|---|---|---|
| 7 | mg | PD (40 | μl) | Na-L lactate | Yes | Clear |

| | |
|---|---|
| (10 | mg) |

| | | | | | | |
|---|---|---|---|---|---|---|
| 7 | mg | PD (40 | μl) | L-lactic | Yes | Turbid |

| | |
|---|---|
| (10 | mg) |

| | | | | | | |
|---|---|---|---|---|---|---|
| 7 | mg | Gly (20 | μl) | PEG/NaAc | Yes | Clear |

| | |
|---|---|
| (20 | μl/10 mg) |

| | | | | | | |
|---|---|---|---|---|---|---|
| 7 | mg | Gly (20 | μl) | PEG/L-lactic | Yes | Turbid |

| | |
|---|---|
| (20 | μl/10 mg) |

| | | | | | | |
|---|---|---|---|---|---|---|
| 7 | mg | PEG (20 | μl) | NaAc | Yes | Clear |

| | |
|---|---|
| (10 | mg) |

| | | | | | | |
|---|---|---|---|---|---|---|
| 7 | mg | PEG (20 | μl) | L-lactic | Yes | Translucent |

| | |
|---|---|
| (10 | mg) |

| | | | | | | |
|---|---|---|---|---|---|---|
| 7 | mg | PD (20 | μl) | benz. SO₃H | Yes | Clear |

| | |
|---|---|
| (10 | mg) |

### Insulin & Aprotinin

| | | | | | | | Addition |
|---|---|---|---|---|---|---|---|
| Insulin/Aprotinin | | | | | | Solu- | of |
| Amount | | | Solvent | | Cosolvent | ble? | Akoline |
| 7 | mg/10 | mg | Gly (40 | μl) | citric acid | Yes | Turbid |

| | |
|---|---|
| (10 | mg) |

| | | | | | | | |
|---|---|---|---|---|---|---|---|
| 7 | mg/10 | mg | PEG (40 | μl) | citric acid | Yes | Turbid |

| | |
|---|---|
| (10 | mg) |

| | | | | | | | |
|---|---|---|---|---|---|---|---|
| 7 | mg/10 | mg | Gly (20 | μl) | PEG/citric acid | Yes | Turbid |

| | |
|---|---|
| (20 | μl/10 mg) |

| | | | | | | | |
|---|---|---|---|---|---|---|---|
| 7 | mg/10 | mg | Gly (40 | μl) | Acetic acid | Yes | Turbid |

| | |
|---|---|
| (10 | mg) |

| | | | | | | | |
|---|---|---|---|---|---|---|---|
| 7 | mg/10 | mg | Gly (20 | μl) | PEG/Acetic | Yes | Turbid |
| | | | | | acid | | |

| | |
|---|---|
| (20 | μl/mg) |

| | | | | | | | |
|---|---|---|---|---|---|---|---|
| 7 | mg/10 | mg | PEG (25 | μl) | DL-lactic acid | Yes | Clear |

| | | |
|---|---|---|
| (25 | μl) | solution |

Gly=glycerol

PG=propylene glycol (1,2-propanediol)

PEG=polyethylene glycol

TEA=triethyl amine

PD=1,3-propanediol

NaAc=sodium acetate

### Biological Testing

Details of the formulations employed in the biological tests described below, are given in Table 2.

Batch No. YNB83108 is a reference batch containing insulin suspended in Akoline; and Batch No. YNBP83109 is a placebo formulation (vehicle only). These are included for comparison.

TABLE 2

| Batch | YNA82801 | YNA82802 | YNB83005 | GNB82101 | YNB83108 | YNBP83109 |
|---|---|---|---|---|---|---|
| Insulin | 7.44 | 7.44 | 7.43 | 7.43 | 7.43 | |
| Solvent system | | | | | | |
| PEG200 | | | | 39.6 | | |
| PEG300 | 30.0 | 30.0 | 24.8 | | | |
| Lactic acid | 30.0 | 30.0 | 24.8 | 9.91 | | |
| Synperonic L44 | | | | 47.2 | | |
| Soy phosphatidyl | | | | 47.2 | | |
| choline | | | | | | |
| Delivery Agent | | | | | | |
| Akoline MCM | 933 | 933 | 943 | | 993 | 1000 |
| Miglyol 810 | | | | 849 | | |
| Total | 1000 | 1000 | 1000 | 1000 | 1000 | 1000 |

All formulations are quoted as mg/dose.

Formulations detailed in Table 2 and used in the biological tests described below were prepared as follows:

Batches YNA82801 and YNA82802 were prepared on a 1 kg scale using the following method:

(a) The solvent system was prepared and the insulin dissolved in the solvent using a magnetic stirrer.

(b) The insulin solution was slowly added to the delivery agent with continuous high shear mixing (Silverson mixer).

(c) The dispersion was dosed to the pigs as a liquid.

Batches YNB83005 and GNB82101 were prepared on a 20 g scale as follows:

(a) The solvent system was prepared and the insulin was dissolved in the solvent using a test tube in a vortex mixer.

(b) The delivery agent and the insulin solution were mixed in a test tube in the vortex mixer.

(c) The dispersion was added to size 1 hard gelatin capsules for dosing to the pigs.

### Intestinal Absorption

The batches, prepared as described above, were tested for their potential to deliver insulin systemically after oral administration of a dose of 200 iu. Oral administration was simulated by application of the dispersions directly to the duodenum of pigs through a catheter and measurement of the increase in insulin over a baseline level measured before insulin application. The data in Tables 3 and 4 below demonstrate that the dispersion formulations can deliver systemic insulin, whereas insulin suspended directly in the mono-and di-glycerides did not appear to deliver insulin. Data in Table 3 are given as the mean (standard deviation) of the individual data from eight pigs (batches YNA82801 and YNA82802) and seven pigs (reference batch

YNB83108). Table 4 provides data from individual pigs.

TABLE 3

| | Batch Reference | | |
|---|---|---|---|
| Measured Property | YNA82801 | YNA82802 | YNB83108 |

| | | | |
|---|---|---|---|
| Insulin maximum increase | 171 | 197 | 18 |
| (μiu/ml) | (198) | (285) | (11) |
| Insulin AUC (h · μiu/ml) | 95 | 70 | 29 |
| | (90) | (142) | (26) |
| Glucose maximum fall | 2.28 | 2.39 | 0.52 |
| (mmol/l) | (119) | (1.40) | (0.31) |
| Glucose AUC (n · mmol/l) | 2.56 | 3.51 | 0.25 |
| | (2.83) | (1.67) | (0.82) |

TABLE 4

| | | | | | | | | | | |
|---|---|---|---|---|---|---|---|---|---|---|
| A | B | C | D | E | F | G | H | K | L | Mean |

Maximum change in glucose level

| | | | | | | | | | | | |
|---|---|---|---|---|---|---|---|---|---|---|---|
| YNB83005 | | −0.42 | −1.01 | −0.23 | −0.72 | −2.96 | | −1.74 | −0.75 | −2.53 | −1.30 |
| GNB82101 | −0.35 | −0.52 | −1.38 | −1.01 | −0.91 | | −0.75 | −0.7 | −0.13 | | −0.72 |
| YNBP83109 | | | −0.18 | | −0.61 | −0.47 | | −0.97 | −0.96 | −0.35 | −0.59 |
| YNB83108 | | | −1.16 | −0.59 | −0.22 | −0.35 | | −0.31 | −0.58 | −0.42 | −0.52 |
| YNA82801 | | −3.33 | −2.95 | −1.01 | −1.20 | −3.24 | | −3.26 | −2.81 | −0.42 | −2.28 |
| YNA82802 | | −1.48 | −3.45 | −0.87 | −1.08 | −4.31 | | −3.15 | −1.07 | −3.72 | −2.39 |

Maximum change in insulin level

| | | | | | | | | | | | |
|---|---|---|---|---|---|---|---|---|---|---|---|
| YNB83005 | | 36.0 | 43.7 | 39.0 | 13.8 | 65.9 | | 16.9 | 49.9 | 53.7 | 39.9 |
| GNB82101 | 15.7 | 31.3 | 5.9 | 5.1 | 5.3 | | −1.6 | 0.1 | 8.4 | | 8.8 |
| YNBP83109 | | | 51.7^{(a)} | | 1.6 | 9.4 | | 13.4 | 6.7 | 3.2 | 14.3 |
| YNB83108 | | | 38.6 | 15.0 | 18.4 | 27.1 | | 7.9 | 8.1 | 9.9 | 17.9 |
| YNA82801 | | 111.0 | 240.2 | 10.2 | 9.7 | 550.5 | | 68.8 | 358.7 | 19.2 | 171.0 |
| YNA82802 | | 91.5 | 829.8 | 3.4 | 11.9 | 166.8 | | 80.2 | 10.3 | 377.9 | 196.5 |

^{(a)}change from baseline reached after 240 minutes; not considered a positive response

1. A pharmaceutical composition comprising insulin and optionally aprotinin, in a substantially non-aqueous hydrophilic medium comprising an alcohol and a cosolvent, in association with a medium chain partial glyceride, optionally in admixture with a long-chain PEG species.

2. A pharmaceutical composition according to claim 1 wherein the alcohol is selected from a C₂-C₈ monohydric alcohol; a C₂-C₈ polyhydric alcohol, or an ether or polyether terminating in one or two hydroxyl groups.

3. A pharmaceutical composition according to claim 1 or claim 2 wherein the alcohol is selected from one or more of ethanol, n-propanol, isopropanol, t-butanol, ethylene glycol (1,2-ethanediol), propylene glycol (1,2-propanediol), trimethylene glycol (1,3-propanediol), glycerol (1,2,3-propanetriol), polyethylene glycol, tetraglycol and transcutol.

4. A composition according to any of claims 1 to 3 wherein the alcohol is polyethylene glycol, tetraglycol or transcutol optionally in admixture with propylene glycol (1,2-propanediol), trimethylene glycol (1,3-propanediol), or glycerol (1,2,3-propanetriol).

5. A composition according to any of claims 1 to 3 wherein the alcohol is propylene glycol (1,2-propanediol), trimethylene glycol (1,3-propanediol), or glycerol (1,2,3-propanetriol).

6. A composition according to any of claims 1 to 5 wherein the liquid polyethylene glycol is selected from PEG 200, PEG 300 and PEG 400.

7. A composition according to any of claims 1 to 6 wherein the cosolvent is selected from an acid; a salt of a weak acid; a weak base; or a zwitterionic compound.

8. A composition according to claim 7 wherein the cosolvent is selected from a carboxylic acid, a sulphonic acid, sodium acetate, sodium ursodeoxycholate, triethylamine and carnitine.

9. A composition according to any of claims 1 to 8 wherein the carboxylic acid is a C₂-C₈ alkylcarboxylic acid, optionally substituted by OH and carrying 1,2, or 3 carboxyl groups.

10. A composition according to any of claims 1 to 9 wherein the carboxylic acid is selected from acetic acid, lactic acid, citric acid, caproic acid and malic acid.

11. A composition according to claim 10 wherein the carboxylic acid is D,L-lactic acid.

12. A composition according to any of claims 1 to 11 wherein the hydrophilic medium comprises an ether or polyether selected from a polyethylene glycol, tetraglycol and transcutol admixed with a C₂-C₈ monohydric alcohol or a C₂-C₈ polyhydric alcohol, and a carboxylic acid or sulphonic acid or a salt selected from sodium ursodeoxycholate and sodium acetate.

13. A composition according to any of claims 1 to 11 wherein the hydrophilic medium comprises an ether or polyether selected from a polyethylene glycol, tetraglycol or transcutol admixed with a C₂-C₈ monohydric alcohol or a C₂-C₈ polyhydric alcohol and a weak base or a zwitterionic compound.

14. A composition according to any of claims 1 to 11 wherein the hydrophilic medium comprises an ether or polyether selected from polyethylene glycol, tetraglycol and transcutol, admixed with a carboxylic acid or sulphonic acid or a salt selected from sodium ursodeoxycholate or sodium acetate.

15. A composition according to any of claims 1 to 11 wherein the hydrophilic medium comprises an ether or polyether selected from a polyethylene glycol, tetraglycol or transcutol, admixed with a zwitterionic compound.

16. A composition according to any of claims 12 to 15 wherein the polyether is a polyethylene glycol.

17. A composition according to any of claims 1 to 11 wherein the hydrophilic medium comprises a C₂-C₈ polyhydric alcohol admixed with a carboxylic acid or sulphonic acid or a salt selected from sodium ursodeoxycholate, sodium acetate and sodium L-lactate.

18. A composition according to any of claims 1 to 11 wherein the hydrophilic medium is selected from:
a polyethylene glycol+1,2-propanediol, 1,3-propanediol or glycerol+acetic acid, citric acid, lactic acid or caproic acid;
a polyethylene glycol+1,2-propanediol, 1,3-propanediol or glycerol+DL-lactic acid;
a polyethylene glycol+1,2-propanediol, 1,3-propanediol or glycerol+triethylamine or carnitine;
a polyethylene glycol+citric acid, lactic acid or caproic acid;
a polyethylene glycol+DL-lactic acid;
a polyethylene glycol+carnitine;
a polyethylene glycol+1,2-propanediol, 1,3-propanediol or glycerol+sodium ursodeoxycholate, or sodium acetate;
a polyethylene glycol+sodium ursodeoxycholate or sodium acetate;
1,2-propanediol, 1,3-propanediol or glycerol+acetic acid, citric acid, lactic acid or caproic acid;
1,2-propanediol, 1,3-propanediol or glycerol+sodium ursodeoxycholate, sodium acetate or sodium L-lactate; and
DL-lactic acid.

19. A pharmaceutical composition comprising a solution of insulin, optionally in admixture with aprotinin, in DL-lactic acid, in association with a medium chain partial glyceride, optionally in admixture with a long-chain PEG species.

20. A composition according to any of claims 1 to 19 wherein the medium chain partial glyceride comprises a mixture of medium chain mono- and di-glycerides.

21. A composition according to any of claims 1 to 20 wherein the medium chain partial glyceride comprises at least 80% of the composition and the hydrophilic medium comprises no more than 20% of the total composition.

22. A composition according to any of claims 1 to 21 wherein the insulin is present in the hydrophilic medium in a concentration of at least 75 mg/ml.

23. A composition according to any of claims 1 to 22 wherein the insulin forms a fine suspension.

24. A method for preparing a pharmaceutical formulation comprising insulin optionally in admixture with aprotinin according to any of claims 1 to 18 and 20 to 23, which method comprises the steps of:
(a) dissolving said insulin and optionally aprotinin in a substantially non-aqueous hydrophilic medium comprising an alcohol, and a co-solvent and
(b) bringing the resulting solution into association with a medium chain partial glyceride, if necessary or desired, in admixture with a long-chain PEG species.

25. A method for preparing a pharmaceutical formulation comprising insulin optionally in admixture with aprotinin according to claim 19, which method comprises the steps of:
(a) dissolving said insulin and optionally aprotinin in DL-lactic acid and
(b) bringing the resulting solution into association with a medium chain partial glyceride, if necessary or desired, in admixture with a long-chain PEG species.

26. Use of a composition according to any of claims 1 to 23 for oral administration.

27. Insulin obtainable by the steps of
(a) dissolving insulin in a substantially non-aqueous hydrophilic medium as defined above;
(b) mixing the resulting solution with a medium chain partial glyceride as defined above;
(c) precipitating a fine suspension of insulin; and
(d) isolating said insulin.

28. Insulin according to claim 27, wherein 50% by weight of the particles have a diameter of less than 30 microns.
